(19) United States
(12) Patent Application Publication
(10) Pub. No.: US 2018/0042262 A1
(43) Pub. Date: Feb. 15, 2018

(54) DENTAL PRODUCT, USE OF A DENTAL PRODUCT AND METHODS OF USE OF A DENTAL PRODUCT

(71) Applicant: DR HEFF'S PRODUCTS LIMITED, EAST SUSSEX (GB)

(72) Inventors: MICHAEL JOSEPH HEFFERNAN, HADLOW DOWN (GB); TOBY EDWARDS-LUNN, HOVE (GB)

(73) Assignee: DR HEFF'S PRODUCTS LIMITED, East Sussex (GB)

(21) Appl. No.: 15/553,718

(22) PCT Filed: Feb. 25, 2016
(86) PCT No.: PCT/EP2016/025018

§ 371 Date: Aug. 25, 2017

(30) Foreign Application Priority Data
Feb. 26, 2015 (GB) 1503211.3

(51) Int. Cl. A23G 4/06; A61Q 11/00; A61K 36/82; A23G 4/08

(52) U.S. Cl. CPC A23G 4/068; A23G 4/08; A61Q 11/00; A61K 36/82; A23G 3/346

(57) ABSTRACT

A dental product (1) including a foodstuff, the foodstuff includes a green tea extract and an amorphous calcium phosphate (ACP). Use of the dental product in a treatment of dental caries. A first method of use of the dental product in the treatment of dental caries including the steps of: consuming a minimum number of dental products in a timeframe of one day (100); spreading the consumption over the timeframe (110). A second method of use of the dental product in the treatment of dental caries including the steps of: consuming the dental product; afterwards, brushing teeth with a toothpaste including a fluoride compound.

### FIELD

The present disclosure relates to a dental product, in particular to a dental product used in the field of caries prevention and therapy, suitable for an older age group. Moreover, the present disclosure concerns a first and second method of using said dental product.

### BACKGROUND

Oral health is an important part of the general health of a person. This field has been under long-term study and advances have been made in various aspects, especially in the treatment and prevention of various kinds of dental decay (e.g. Vidal et al, Acta Biomaterialia 10, 3288-94, 2014). Such progress is widely applicable to all ages and to varying geographical territories.

In particular, work has highlighted the issue of aging populations. Examples comprise: Walls and Meurman (Adv. Dent. Res. 24, 2, 36-40, 2012); Narhi et al (Drugs Aging 15: 103-116; 1999) and Cochrane and Reynolds (Adv. Dent. Res. 24, 2, 41-47, 2012). Some oral health issues are more pronounced for this group, which is frequently characterised by a population aged 65 or more. Due to increase in life expectancy, the maintenance of oral health becomes more important and more challenging for this group. An aging person often faces extra problems due to age-specific problems, such as e.g. polypharmacy (the taking of four or more different medications), reduced ability to maintain oral hygiene (physical limitation), dry mouth or xerostomia (due to the natural aging process) etc.

Root caries has been identified as a common cause of tooth loss in an older population. However, this problem is not exclusive to such a population. Various studies have been carried out to identity methods which can be used to prevent and treat the problem e.g., Gluzman et al (Special Care in Dentistry, 33, 5, 218-26, 2013), and Ritter et al (J Dent Res 92, 6, 512-17, 2013). Remedies include the daily use of fluoride toothpastes with special anti-bacterial ingredients, such as triclosan, or compounds such as ACP (amorphous calcium phosphate).

Another option identified was a professionally applied sodium fluoride (NaF) varnish, with the application repeated every 1-3 months. The concentrations of active ingredients vary across these treatments—e.g., NaF in the varnish is quoted as present to a level of 22,500 ppm, whereas in the toothpaste the level is around 4-5,000 ppm. In addition, other chemical compounds and ingredients were tested, with silver diamine fluoride presenting as an effective equivalent to sodium fluoride in the varnish, as did chlorohexidine.

Xylitol, a sweetening agent with an anticariogenic effect, was identified as beneficial to reduce caries and as working in co-operation with fluoride toothpaste to produce additional benefit. Xylitol is a naturally occurring non-fermentable polyol used as a sugar substitute, and is therefore considered a non-cariogenic sweetener. A study by Ritter et al, published in the Journal of Dental Research, J DENT RES 92: 512 (April 2015), evaluated the effectiveness of xylitol vs. placebo lozenges in the prevention of dental caries in caries-active adults. Dental caries is defined as resulting from an imbalance of de-mineralization and re-mineralization processes at the tooth-biofilm interface over time. The purpose of this study was to compare the caries-preventive effects of xylitol (1) on root and coronal surfaces and (2) among coronal surfaces, on smooth (buccal and lingual), occusal, and proximal surfaces. Intervention comprised five xylitol lozenges. Spaced across the day and dissolved in the mouth. Results showed no statistically significant difference between xylitol and control participants in the incidence of smooth-surface caries, occusal-surface caries or proximal-surface caries. Results from previous research are also quoted as suggesting xylitol could have a tooth-surface-specific caries-preventive effect. However, xylitol appears to have a caries-preventive effect on root surfaces. The participants in the study lived in areas where fluoride was added to water and this leads to the possibility that the results may be due to xylitol being an add-on intervention. Further, it is suggested, based on previous research, that the root surface caries is mostly a dentine caries phenomenon and that it is plausible that xylitol exerts a higher caries-preventive effect at the pH of dentin demineralization than that of enamel. Overall, the conclusion is given that xylitol lozenges, given their low cost, low risk and ease of use, do help to prevent root caries.

In the presence of an acidic environment calcium and phosphorous ions are stripped from the tooth substance in a process of demineralisation. However, this is a reversible process and amorphous calcium phosphate (ACP) can be used to tip the equilibrium in favour of re-uptake of calcium and phosphorous ions into the tooth (remineralisation) (Tung et al, Compendium; 25, 9,9-13, 2004). ACP is an essential mineral phase formed in mineralised tissues. The amorphous phase of the material results in a highly reactive compound, which is more soluble in plaque. The effectiveness of this compound is enhanced by its generally good biological properties and biodegradability.

Green tea extract, comprising various polyphenols, is purported to have benefits to general health through its antioxidant properties and therefore is considered anti-mutagenic, anti-carcinogenic, and improvements in cardiovascular risk (Narotzki et alArchives of Oral Biology, 57, 5, 429-35, 2012). In particular, a polyphenol component, eGCG (epigallocatechin gallate), has been shown to have an antibacterial effect, an anti-mutagenic effect, and a favourable effect on blood cholesterol level. It is also a MMP (matrix metalloproteinases) inhibitor and has been found to increase the modulus of elasticity and reduce the biodegradation rate of dentine by increasing the collagen stabilisation, therefore it can structurally modify and improve dentine tissue (Vidal et al 2014).

The eGCG component, a type of catechin found in green tea extract, is used as active ingredient of anti-periodontal disease treatments. Such molecules are advantageous in their lack of undesirable side effects, when compared with synthetic drugs, and are therefore compatible with treatments for dentine caries. The specific composition of green tea extract products is highly dependent on supplier but data on the composition is normally available to a purchaser. For example, a data sheet for one green tea extract, available for sale, lists the product as comprising 40% polyphenols, 20% catechins and 10% eGCG.

The methods and materials comprised in the prior arts are limited in their effectiveness in the treatment of root caries and associated tooth loss.

### SUMMARY

The present disclosure seeks to provide an improved dental product. Moreover, the present disclosure seeks to provide a dental product optimised to the needs of an older age group.

According to a first aspect of the present disclosure, there is provided a dental product: there is provided a dental product comprising a foodstuff, the foodstuff comprising:

a green tea extract between 2% and 10% by weight, wherein wherein the green tea extract comprises eGCG between 5% and 60%; and

an amorphous calcium phosphate (ACP), wherein the foodstuff comprises between 1% and 10% amorphous calcium phosphate by weight.

The disclosure is of advantage in that it provides a synergistic product comprising the oral health advantages of green tea and ACP in an easy to consume form. To promote remineralisation the eGCG may protect the dentine matrix from enzymatic degradation and the ACP may provide the ions to replace those lost in the demineralisation phase. This is a particularly easy way of obtaining these important ingredients for older people to assist in the treatment of root caries and improvement of oral health. The form of the product encourages retention in the mouth, thereby providing contact time for the ingredients and chemical action.

Optionally, the green tea extract comprises 40% eGCG.

Optionally, the foodstuff comprises 1% ACP by weight.

Optionally, the foodstuff may further comprise xylitol, wherein the foodstuff comprises between 50% and 95% of xylitol by weight.

Optionally, the foodstuff comprises 80% and 95% of xylitol by weight, wherein the foodstuff comprises 94.5% of xylitol by weight.

Optionally, the foodstuff further comprises a flavouring, such as a mint or fruit flavouring, comprising natural and/or artificial ingredients, even more preferably a mint flavouring comprising natural peppermint oil and/or menthol.

Advantageously, this makes the product more palatable to the consumer.

Optionally, the foodstuff comprises between 0.1% and 2% natural peppermint oil by weight, even more preferably around 1% natural peppermint oil by weight.

Optionally, the foodstuff comprises between 0.1% and 2% menthol by weight, even more preferably around 1% menthol by weight.

Optionally, the foodstuff comprises calcium stearate and/or magnesium stearate, between 0.5% and 1%, preferably around 0.5% by weight.

Advantageously, the addition of one or both of these compounds, i.e. stearate and/or magnesium stearate, provides a binding or caking effect for an embodiment of the invention comprising a foodstuff in a solid form.

Optionally, the foodstuff comprises a form of a lozenge, pastille, sweet, gum or liquid drink, wherein the form is selected for allowing active ingredients to linger in the mouth for an extended period.

According to a second aspect of the invention disclosure, there is provided a use of a dental product in a treatment of dental caries and improvement of oral health, wherein the dental product is used immediately before using a fluoride containing toothpaste for enhancing the fluoride uptake of fluoride from the toothpaste.

According to a third aspect of the invention disclosure, there is provided a first and a second method of use of a dental product. The composition of the dental product as described above.

A first method of use of the dental product in the treatment of dental caries comprises the steps of; consuming a minimum number of dental products in a timeframe of one day; and spreading the consumption over the timeframe.

Advantageously, this allows a desired intake of dental product to be achieved over a specified timeframe, e.g. one day.

Optionally, the method includes implementing the spreading periodically over the timeframe.

Advantageously, this provides a regular and consistent dose of active ingredients over the timeframe.

A second method of use of the dental product in the treatment of dental caries comprises the steps of: consuming the dental product; afterwards, brushing teeth with a toothpaste comprising a fluoride compound.

Advantageously, the use of the dental product according to an embodiment of the present invention disclosure immediately before the use of a fluoride containing toothpaste allows the presence of a high concentration of calcium and/or phosphorous in the mouth (or in the saliva), which can enhance the fluoride uptake from the toothpaste.

In the event that the toothpaste is not a fluoride containing product, use of a dental product according to the present invention disclosure continues to provide oral health benefits. For users with no access to fluoride, either in toothpaste or in water for example, these benefits have heightened importance.

Although the dental product is particularly suitable for use by an older age group, there is no embargo on use by people of other ages. Dosage may be adjusted as desired for other age groups, depending on the particular needs of the age group.

It will be appreciated that features of the disclosure are susceptible to being combined in any combination without departing from the scope of the invention as defined by the appended claims.

### DESCRIPTION OF THE DIAGRAMS

Embodiments of the present invention disclosure will now be described, by way of example only, with reference to the following diagrams wherein:

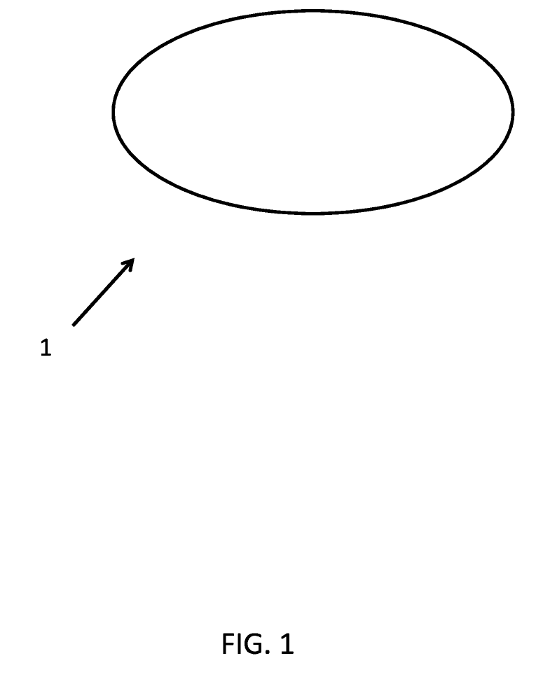

FIG. 1 is an illustration of a dental product according to an embodiment of the invention disclosure, top view;

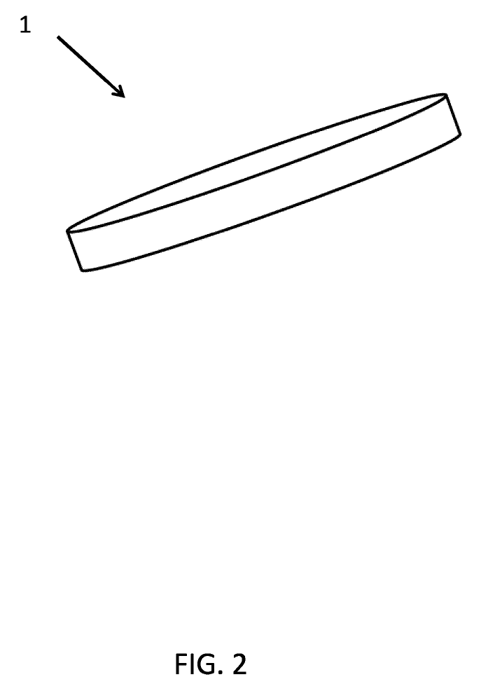

FIG. 2 is an illustration of a dental product according to an embodiment of the invention disclosure, tilted side view;

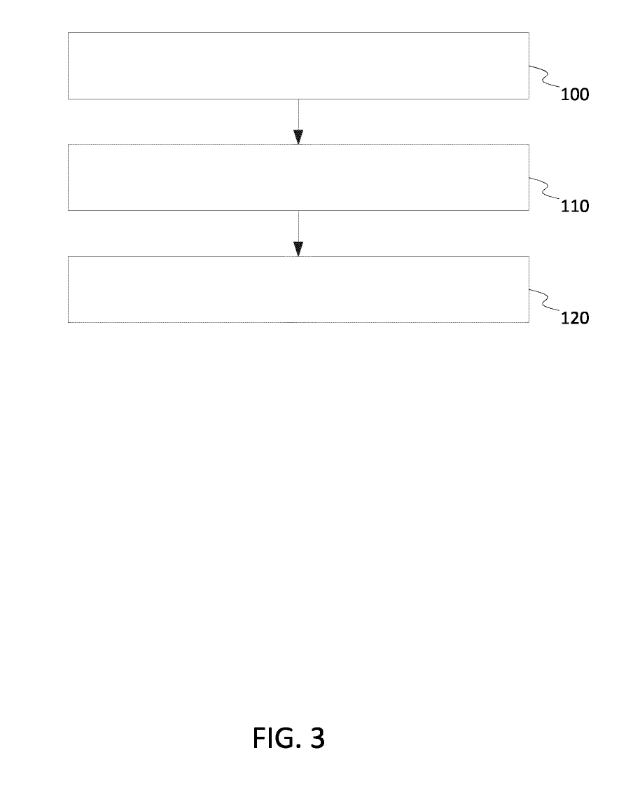

FIG. 3 is an illustration of steps of a method of use of the dental product of FIG. 1 and FIG. 2;

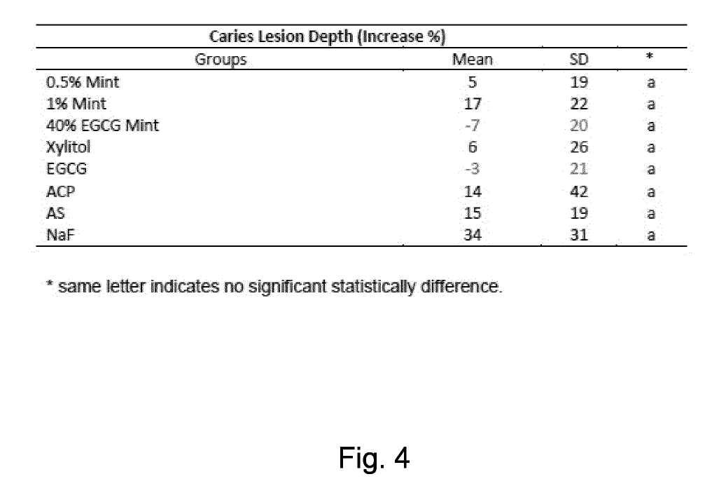

FIG. 4 is a table of experimental results, comprising data on caries lesions after pH-proteolytic cycling and treatments expressed as percentages (%) of increase/decrease from initial lesions, wherein the same letter in column * indicate no significant statistical difference;

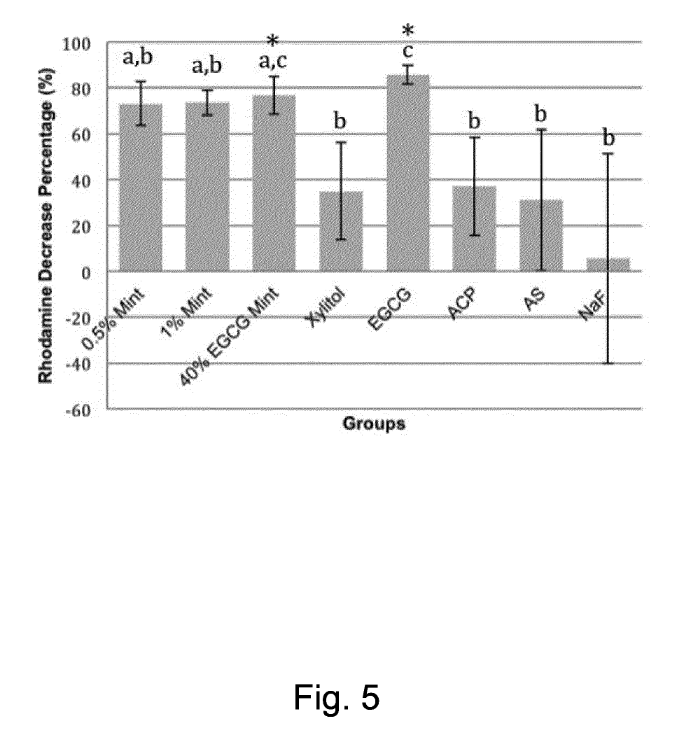

FIG. 5 is a chart representing experimental data, comprising a decrease percentage (%) of the Rhodamine intensity after pH-proteolytic cycling and treatments; and,

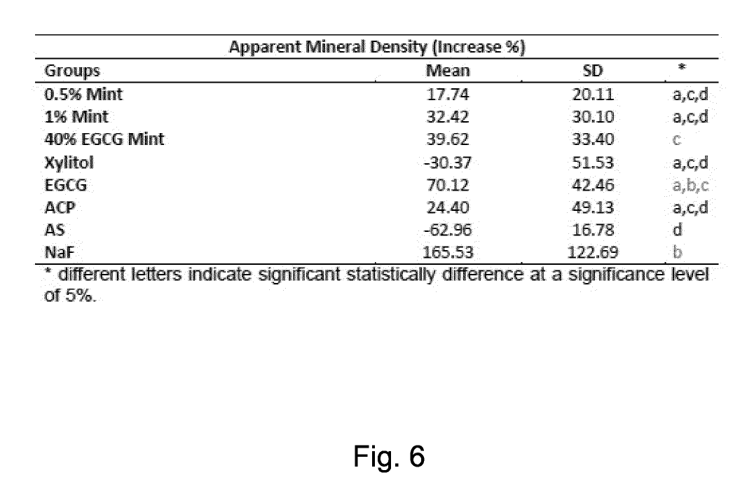

FIG. 6 is a table of experimental results comprising data on the increase percentage (%) of the apparent mineral density (mg HA/ccm) after pH-proteolytic cycling and treatments, wherein, the different letters in column * indicate a statistical different at a significance of 5%.

### DESCRIPTION OF EMBODIMENTS

In FIG. 1 and FIG. 2, a dental product according to an embodiment of the present invention disclosure is shown as generally represented by the numeral 1. The dental product is shown in the form of foodstuff comprising a sweet, which is oval in shape. The sweet has a designed depth, narrow compared to the area of its face. Such a surface area to volume ratio is advantageous for quick dissolution of the sweet in the mouth. This further intensifies the concentration of dental product components present in the mouth at a particular moment.

The sweet may be shaped in any desired form e.g., circular, square etc. The oval shape pictured here is not intended to be in any way limiting. While the depth is not critical, a thinner sweet possesses the advantages as discussed above.

Further, while the sweet shown has a smooth surface, the sweet may be embossed with a pattern or name marking for easy recognition.

The foodstuff may also comprise a chewing gum, sweet gum, drink etc. That is to say, the product is not restricted to a solid form, but may also take the form of a liquid. For the best effect in use, the product should be presented in a form that allows the active ingredients to linger in the mouth for an extended period, as opposed to being quickly chewed and swallowed. The product is, preferably, flavoured so that the consumer finds the product pleasant to take and is motivated to use it. Natural flavourings are preferred due to the health benefits provided; particularly natural peppermint oil is favoured, as it is familiar to consumers and is popular. Another ingredient with a strong flavour, which is suitable for incorporation, is menthol. A caking agent, such as calcium or magnesium stearate, may also be incorporated in the dental product, but is not an active ingredient for the purpose of dental hygiene.

In a particular embodiment of the present invention, the foodstuff comprises a sweet flavoured by a natural mint flavour, for enhanced acceptability to the consumer. The sweets or mints are provided in a tablet of weight 0.66 g (660 mg) made with the following ingredients: xylitol (94.5%, 623.7 mg); amorphous calcium phosphate (ACP) (1.0%, 6.6 mg); green tea extract (2.0%, 13.2 mg); natural peppermint oil (1.0%, 6.6 mg); calcium stearate (0.5%, 3.3 mg), menthol (1.0%, 6.6 mg).

Alternative ingredients include grape seed extract as a replacement or supplement for eGCG and other forms of calcium phosphate, such as brushite for ACP.

Referring to FIG. 3, the dental product 1 is used in a manner as defined by steps of a method whose steps are illustrated. In a first step 100, the method comprises consuming a minimum number of dental products in a timeframe of one day. A second step 110, of spreading the consumption over the timeframe, allows the correct target amount of ingredients to be consumed. Optionally, a third step 120 of implementing the spreading periodically over the timeframe, gives the possibility to present a steady and regular input of the dental product.

The recommended daily amount of dental product is dependent on the actual implementation of the invention disclosure. In the example case of the sweet 1, the overall weight of the sweet (e.g. 660 mg per sweet) will comprise a certain amount of active ingredients, such as green tea extract and ACP, to a specific percentage by weight. Targeting the problem of dental caries, the use of the dental product can be rendered optimally effective when an amount of the product consumed delivers at least, or more than, a target daily amount of specific ingredients. For example, the delivered amount of ACP may comprise 1.4 milligram (mg) per serving (i.e. per sweet, for example), but would be efficient in an amount between 1 mg and 50 mg per day. A recommended amount of xylitol per day ranges between 3 g to 6 g, which may be split between sweets over the day, if desired. Green tea extract as a raw material varies in the amount of eGCG present. Depending on the composition of the extract, an eGCG dosage can be incorporated in the sweet by adjusting the amount of green tea extract added. If the green tea extract comprises 10% eGCG, then 66 mg green tea extract would provide at least a target amount (6.1 mg) of eGCG per serving or e.g. sweet.

The processing and manufacture of the foodstuff, e.g. the sweet, may dictate the overall size of the finished product. This, in turn, may limit the amount of ingredients available by the consumption of one serving of the foodstuff, e.g. one sweet. Naturally, this can be compensated in the usage instruction and two or more items of the foodstuff can be consumed simultaneously, if desired. The foodstuffs can be manufactured by known methods and by combination of the appropriate ingredients, according to various embodiments of the invention.

Referring now to FIG. 4 and subsequent figures, details of experimental work on a dental product in the form of a sweet, according to embodiments of the present invention, will be presented. Experimental details are provided in Appendix A.

In a particular embodiment of the present invention associated with the experimental work, the eGCG content of a foodstuff in the solid form, is dependant on the content of a green tea extract of a sweet. The eCGC content in green tea is around 40% by weight. The eCGC content in green tea may vary from around 5% to around 80% by weight. Therefore, a particular and preferred green tea extract is used to achieve this percentage. Different green tea extracts contain different amounts of eGCG, and this can be used to tailor the dental product as desired. The desired percentage, preferably 40%, may, for example, be achieved by combining different green tea extracts. Here 40% refers to the purity of the green tea extract and not the overall weight of the sweet itself. (In the examples to be presented below, this amount may correspond to 40% of a total 13.2 mg of green tea, per sweet, in this case a mint flavoured sweet). A refinement of the green tea extract, or a different choice of extract, can be used to achieve eGCG percentages up to around 80%.

Experimental work concentrated on this particular composition of product but the product component details, general conclusions and results are broadly applicable across the various embodiments of the present invention disclosure.

The reagents used for the production of the dental product, according to embodiments of the present invention disclosure, are intended to comprise the ACP, which provides calcium phosphate in a highly reactive form, which will promote crystallisation of hydroxyapatite on a tooth surface to prevent the formation of caries in dentine or repair the initial stages of cavity formation. For embodiments of the present invention disclosure provided in solid form, at least the raw ACP material is preferably presented in the form of a powder for processing into a tablet product. The ACP is preferably in the amorphous form, rather than crystalline, and predominantly comprising hydroxyapatite together with other calcium phosphate minerals. Elements present in the powder comprise, for example, but not limiting to, calcium, oxygen, phosphorous, and carbon.

Preferably, the ACP is distributed in discrete areas of the tablet surface. The ACP remains as a separate component within the tablet matrix following the tableting procedure. Further, the ACP is preferably provided as an ingredient in the form of a 1.5:1 powder. The ACP 1.5:1 powder remains within the matrix of the tablet in a granular form in discrete granules, similar to those seen in the original powder. The distribution of carbon and oxygen suggests the bulk of the matrix of the material consists of the organic components of the tablets (xylitol and green tea extract). The ACP powder may contain a non-crystalline component. The non-crystalline component remains as a relatively minor component of the calcium phosphate contribution to the tablet, which itself is present as discrete areas within the organic matrix of the tablet consisting of xylitol and green tea extract.

The tablet forming process (e.g. pelleting) is preferably arranged to permit the nature and composition of the ACP to remain consistent with the raw material, where the ACP remains within the matrix of a tablet in a granular form in discrete granules similar to those of the original powder. Preferably, the ACP remains as a separate component within the tablet matrix following the tableting procedure, further distributed within discrete areas of the tablet surface. A preferred ACP powder comprises, at least in part, a nano-crysalline nature, to effect a similar reactivity to a truly amorphous material. The nano-crystalline component may be a relatively minor component of the calcium phosphate contribution to a tablet, which itself is present as discrete areas within the organic matrix of the tablet comprising xylitol and green tea extract. For example, in a particular embodiment of the present invention disclosure, a tablet comprised an average crystallite size of around 42 nm (nanometre). A mean crystallite diameter of between 40 nm and 50 nm for nano-crystalline hydroxyapatite, dominant in the ACP composition is preferred. Some recrystallization of the ACP powder during the pelleting process can trigger crystallite sizes up to 0.15 μm (micrometres) or 150 nm. The mineralogical changes are unlikely to affect the formation of hydroxyapatite on dentine, as the phosphates are intended to undergo a dissolution and re-precipitation process in mouth saliva. The crystalline coarsening may have some impact on the tablet in use but the sizes are still small enough to give a very high surface area, which would enhance the reactivity of the Ca-phosphate (calcium phosphate) phases. The surface of the tablet at high magnification (e.g. 200 000X) therefore comprises a combination of smooth areas, consistent with organic components (xylitol and green tea extract, for example) of the formulation, and roughened areas. Discrete areas of crystalline material are also present, consistent with the nano-sized features of the ACP powder.

In particular, root decay is known to be caused by fermentation of carbohydrates by bacteria, which may remove the minerals from the root (particularly calcium and phosphates). The collagen structure of the root is then susceptible to being broken down by enzymes (e.g. collagenases). Once this happens there is no way to rebuild the structure of the tooth, other than by dental restorations. The preservation of the collagen scaffold is key, so by the use of eGCG, as comprised in dental products according to embodiments of the present invention disclosure, the scaffold can be maintained to allow the ACP to re-mineralise and the xylitol to limit the bacteria that cause decay.

In addition, tooth erosion of enamel and dentine occur in a similar way but without the bacterial production of acids. Dental products, according to embodiments of the present invention disclosure, are also effective in the prevention of erosion on tooth surfaces.

These research studies, detailed in Appendix A with main results presented below, have been conducted during the course of product development. The experiments comprise comparisons with other types of product of differing composition to gauge the effectiveness of various product components with respect to the clinical issues outlined above. These studies, using xylitol alone (with artificial saliva), have shown that dental tissue loss occurs, which could be due to the loss of the collagen scaffold and consequent loss of structure. Advantageously, the use of combined ACP and eGCG compounds with xylitol overcome this problem. In addition, the studies have shown that the xylitol alone (with artificial saliva) does not help to increase mineral density, whereas the ACP does increase mineral density in the caries lesion. In particular, the ACP in the form described above is advantageous. The ACP and the green tea may allow both re-mineralisation and preservation of tooth substance. Dental products according to embodiments of the present invention disclosure are also more effective than the xylitol only products at re-mineralisation of dentine.

Advantageously, the inclusion of the ACP and the xylitol does not inhibit the effect of eGCG. The eGCG is effective at preserving dentin matrix (even compared with fluoride, which is typically regarded as leading in this respect) and eGCG promotes mineral density similar to fluoride and better preservation of tooth substance i.e., less porosity.

Specific details about said experimental results, concerning the dental product according to embodiments of the present invention disclosure, are discussed below. In one set of results, fluoride was used, as a control. Fluoride is a well-known ingredient in many dental products, effective against decay.

An objective of the experimental study was to investigate the effects of mints containing bioactive agents, specifically the xylitol, ACP and eGCG, in the re-mineralization of root dentin surfaces. In vitro pH-proteolytic cycling modelling was used to demonstrate the de-mineralization and re-mineralization potential of experimental product targeting the mineral and organic components of dentin. The caries lesions depths and porosity as well as mineral density (hydroxyapatite content) were estimated using fluorescence microscopy and micro-computed tomography.

Different experimental samples comprising differing compositions. A preferred embodiment of the present invention disclosure, is known here as the 1% mint. This is because it comprises a 1% level of ACP. Specifically, the components of the 1% mint are provided in a tablet of weight 0.66 grams or 660 mg made with the following ingredients: xylitol (94.5%, 623.7 mg); ACP (1.0%, 6.6 mg); green tea extract (2.0%, 13.2 mg); natural peppermint oil (1.0%, 6.6 mg); calcium stearate (0.5%, 3.3 mg), menthol (1.0%, 6.6 mg). (This composition is the same as the embodiment of the present invention disclosure as described in FIG. 1, which can comprise various percentages of eGCG in the green tea extract, 10% being used as an example in the text associated with FIG. 1). For the so-called 0.5% mint or sweet, another preferred embodiment of the present invention disclosure, the ingredients and sizing are the same but the level of ACP is reduced to 0.5% (3.3 mg/sweet), with a consequent increase in xylitol percentage to 95% (627 mg/sweet).

Other experimental substances included a 40% eGCG mint, comprising an eGCG component of specifically 40% of the green tea extract (itself comprising 2% of the overall weight of the sweet, as above). In addition, concentrations of substances comprising xylitol only, eGCG only, and ACP only were arranged to be applied, in order to investigate effects of the individual components.

The mints and other substances, comprised into groups, were applied to the dental samples by suspending all ingredients and mints in suspension of artificial saliva (20 mM HEPES; 2.25 mM CaCl2·2H2O; 1.35 mM KH2PO4; 130 mM KCl, pH 7.0) at concentrations of 0.22 g/0.5 mL, 0.22 g/0.5 mL, 0.22 g/0.5 mL, 0.2079 g/0.5 mL, 4.4 mg/0.5 mL and 2.2 mg/0.5 mL for 0.5% Mint, 1% Mint, 40% eGCG Mint, Xylitol, eGCG and ACP, respectively. A NaF (sodium fluoride) control was presented at 1000 parts per million (PPM) level.

Referring now to FIG. 4, FIG. 5 and FIG. 6, which depict experimental data for the studied groups as outlined above.

FIG. 4 shows a table of experimental results, comprising data on caries lesions after pH-proteolytic cycling and treatments expressed as percentages (%) of increase/decrease from initial lesions. Significant (% caries lesion depth) decrease results are shown for the 40% eGCG mint and the eGCG only groups.

Results were obtained from fluorescence microscopy. The initial caries lesions were successfully obtained after 96 h using a de-mineralization solution (chemical model) at low pH (4.6). The mean depth of the initial lesions was 123±23 micrometre (μm), as measured by Rhodamine and DIC images. After treatments and pH-proteolytic cycling for 8 days, the mean depth of the final lesions for all samples was 132±28 μm with different patterns of caries progression. The depth of the caries lesions is presented as percentage change (FIG. 4). The result of the Levene's test (p=0.275) confirmed the homogeneity of variances. Data was analyzed by ANOVA test (α=0.05). No statistically significant differences were observed among all groups (p>0.05).

FIG. 5 is a chart representing experimental data, comprising a decease percentage (%) of the Rhodamine intensity after pH-proteolytic cycling and treatments, where * indicates significant statistical difference compared to AS group. Different letters indicate significant statistical difference.

The decrease percentage of the mean fluorescence (F) exhibited by caries lesions revealed significant statistically differences among groups (FIG. 5). The highest F decrease percentage (85.72%) was observed for eGCG group followed by 40% eGCG Mint (76.78%) without statistically significant difference among them. It was possible to observe statistically significant differences among eGCG (p=0.017) and 40% eGCG Mint (p=0.043) groups compared to AS (negative control). Although there were numeric differences among 0.5% Mint (p=0.067) and 1% Mint (p=0.061) groups compared to AS group, these differences did not reach statistical significance. Similar reductions without significant statistically difference (p>0.05) on the F values were obtained for ACP, xylitol, AS and NaF groups with percentage of 37%, 34.92%, 30.99% and 5.60%, respectively.

Dental tissue loss was mainly observed on the xylitol and AS groups.

FIG. 6 is a table of experimental results comprising data on the increase percentage (%) of the apparent mineral density (mg HA/ccm) after pH-proteolytic cycling, and treatments.

Results were obtained using micro-computed tomography. The increase percentages in the apparent mineral density in all groups were statistically analyzed by analysis of variance (ANOVA) and Tukey test at α=0.05. The results are presented on FIG. 6. The highest increase percentage of the apparent mineral density was observed for the NaF group followed by eGCG and 40% Mint groups with mean values of 165.53%, 70.12% and 39.62%, respectively. No significant statistical difference was found between NaF and eGCG groups (p=0.084) and eGCG and 40% eGCG groups (p=0.979). These groups are statistically significant higher than AS group (p<0.05). No significant difference was observed among other groups (p>0.05).

Although a re-mineralization trend was observed, mints containing lower concentration of eGCG were not statistically different than control (AS). Catechins are calcium binding, and therefore eGCG act as phosphoprotein analogue to nucleate mineral formation in collagen scaffold in dentin.

### Experimental Conclusions:

Within the limitations of this in vitro pH-proteolytic cycling study, the following conclusions can be drawn:
There was no significant effect of the experimental treatments on the lesion depth of artificially induced root dentin caries.
Re-mineralization of artificially induced dentin caries was observed by significant decrease in lesion porosity of specimens treated with 40% eGCG Mints and eGCG regimens.
Re-mineralization was confirmed by increased mineral density in the 40% eGCG Mints and eGCG groups when compared to control (AS).
ACP and Xylitol alone had no significant effect on the caries re-mineralization.
Mints containing high concentration of eGCG, shows re-mineralization potential on artificially induced root caries. The eGCG is an active ingredient in the Mint formulations.

Overall, a preferred embodiment of the present invention disclosure includes a dental product in the solid form of a sweet, optionally flavoured with mint, which comprises a combination of eGCG comprised in green tea extract, xylitol and ACP. The level of eGCG is preferably arranged in a range between 35% and 45% or between 40% and 80%, but most preferably as 40%, of the green tea extract.

Although embodiments of the present disclosure are described in the foregoing, it will be appreciated that the present invention disclosure is also susceptible to being implemented in many embodiments, depending on the particular application and implementation of form.

Modifications to embodiments of the present disclosure described in the foregoing are possible without departing from the scope of the invention as defined by the accompanying claims. Expressions such as “including”, “comprising”, “incorporating”, “consisting of”, “have”, “is” used to describe and claim the present invention disclosure are intended to be construed in a non-exclusive manner, namely allowing for items, components or elements not explicitly described also to be present. Reference to the singular is also to be construed to relate to the plural. Numerals included within parentheses in the accompanying claims are intended to assist understanding of the claims and should not be construed in any way to limit subject matter claimed by these claims.

### APPENDIX A

Study Aims

The objective of this study was to investigate the effects of mints containing bioactive agents (e.g. xylitol, ACP and eGCG) in the re-mineralization of root dentin surfaces. In vitro pH-proteolytic cycling model was used to demonstrate the de- re-mineralization potential of experimental product targeting the mineral and organic components of dentin. The caries lesions depths and porosity as well as mineral density (hydroxyapatite content) were estimated using fluorescence microscopy and micro computed tomography.
Materials and Methods

Extracted sound bovine incisors were freshly collected from a slaughterhouse, cleaned and stored at 4 oC in 0.1% thymol for no longer than 2 weeks. Two root fragments (5×4×1 mm) from the cervical portion (mesial and distal) of the root were obtained from each tooth using diamond disk mounted in a cutting machine (Isomet 1000, Buehler, Lake Bluff, Ill., USA). The dentin surfaces were polished using #600 and #800 abrasive paper (Carbimet, Buehler) under constant water irrigation. The entire surface of the fragments was sealed with acid-resistant nail polish, except for a 4×3 mm window on the polished dentin surface.
Artificial caries lesion formation:

The root fragments were placed in a demineralization solution (2.2 mM CaCl2·2H2O, 2.2 mM KH2PO4, 50 mM acetate, pH 4.6) for 96 h at 37° C. to create initial caries lesions of approximately 120 μm-deep (1). Following lesion development, the fragments were rinsed thoroughly with deionized water, and half of the specimens' windows were covered with an acid-resistant nail polish to maintain the baseline lesion.
Experimental Therapies:

The root fragments were randomly divided into eight experimental groups (n=8 samples per group) including a negative control (artificial saliva, AS) and positive control (1000 ppm NaF), as described in above text. Specifically: 0.5% mint, 1% mint, 40% eGCG mint, xylitol, eGCG, ACP, AS, NaF.

All ingredients and mints were suspended in artificial saliva (20 mM HEPES; 2.25 mM CaCl2·2H2O; 1.35 mM KH2PO4; 130 mM KCl, pH 7.0) at concentrations described in Table 2. The concentrations were optimized to simulate the in vivo conditions in which the normal salivary flow is 0.5 mL/min (2). Thus, one third of the mints (g) were dissolved in 0.5 ml of artificial saliva and the ingredients concentrations were kept at same ratio as in the original mint formulation.
De-remineralization Regimen (pH cycling model):

All samples were pH-cycled through treatment solutions (5 times per day for 3 minutes), and 6 cycles of acidic buffer (50 mM acetate; 2.25 mM CaCl2·2H2O; 1.35 mM KH2PO4; 130 mM KCl; pH 5.0; 30 min), and neutral buffer (20 mM HEPES; 2.25 mM CaCl2·2H2O; 1.35 mM KH2PO4; 130 mM KCl, pH 7.0; 10 min) for 8 days (1). All solutions were prepared daily prior to use. In addition to pH cycling, the overnight re-mineralization solutions had bacterial collagenase from Chlostridium Histolycum (100 μg/ml) to simulate enzymatic challenge.
Post-treatment analysis:

Each specimen was sectioned perpendicularly to the varnished area so that each section included the baseline and post-treatment lesions. Half of each sample was used for fluorescence microscopy and the other half was used for micro-computed tomography.
Fluorescence Microscopy:

Samples were sectioned (n=8), polished and stained with a freshly prepared 0.1 mM Rhodamine B solution (Aldrich Chem. Co., Milwaukee, Wis., USA) overnight, and rinsed for 1 min with deionized water (3). Samples were analyzed with a Fluorescence Microscope (DMI6000 B, Leica), using TEX and DIC (differential interference contrast) channels. Fluorescence intensity line profiles obtained in TEX channel images of the pre and post-treatment lesions were analyzed in Image J Software (NIH), thereby providing mean intensity values (per pixel) for red emission channel of each image. The fluorescence intensity is directly related to the porosity of the de-mineralized dentin, where increased porosity is directly related to increased fluorescence intensity. Fluorescence data was presented as percentage difference from post-treatment and pre-treatment lesions of each specimen. Additionally, the depths of caries lesion (initial and final lesions) were calculated and data presented as percentage increase/decrease of post-treatment lesions to initial lesions (baseline).
Mineral density using micro computed tomography (μCT)

Specimens (n=6 per group) were fixed in 10% neutral buffered formalin for 24 h. Specimens were held securely in a specimen holder, which were soaked with phosphate-buffered saline (PBS). The scanning parameters were 55 kVp energy (X-ray voltage), 109 μA intensity, and 500 ms integration time using 3.4 μm voxels/resolution, with a 1024×1024 reconstructed image (μCT-50, Scanco Medical, Wayne, Pa., USA). The manufacturer's software was used to filter noise and segment the data at a threshold of 220. Tissue mineral density (based on a standard calibration phantom provided by the manufacturer) was determined (mg/cc of hydroxyapatite). Three-D imaging of the mineralization pattern was used for spatial distribution of mineral within the lesions in an area of 100×10 pixels, including 200 slices.
Data and Statistical Analysis:

The assumption of equality of variances and normal distribution of errors were verified using Levene's test. Analysis of variance (ANOVA) followed by Scheffe or Game-Howell tests were performed at α=0.05 using SPSS 20 software.

1-15. (canceled)

16. A dental product (1) comprising a foodstuff, the foodstuff comprising:
a green tea extract between 2% and 10% by weight, wherein wherein the green tea extract comprises eGCG between 5% and 60%; and
an amorphous calcium phosphate (ACP), wherein the foodstuff comprises between 1% and 10% amorphous calcium phosphate by weight.

17. A dental product as claimed in claim 16, wherein the green tea extract comprises 40% eGCG.

18. A dental product as claimed in claim 16, wherein the foodstuff comprises 1% amorphous calcium phosphate by weight.

19. A dental product as claimed in claim 16, wherein the foodstuff further comprises xylitol, wherein the foodstuff comprises between 50% and 95% of xylitol by weight.

20. A dental product as claimed in claim 19, wherein the foodstuff comprises between 80% and 95% of xylitol by weight, wherein the foodstuff comprises 94.5% of xylitol by weight.

21. A dental product as claimed in claim 16, wherein the foodstuff further comprises a flavouring, preferably a mint or fruit flavouring comprising natural and/or artificial ingredients, even more preferably a mint flavouring comprising natural peppermint oil and/or menthol.

22. A dental product as claimed in claim 21, wherein the foodstuff comprises between 0.1% and 2% natural peppermint oil by weight, even more preferably around 1% by weight.

23. A dental product as claimed in claim 21, wherein the foodstuff comprises between 0.1% and 2% menthol by weight, even more preferably around 1% by weight.

24. A dental product as claimed in claim 16, wherein the foodstuff comprises a calcium stearate and/or a magnesium stearate, preferably around 0.5% by weight.

25. A dental product as claimed in claim 16, wherein the foodstuff comprises the form of a lozenge, a pastille, a sweet, a gum, or a liquid drink, wherein the form is selected for allowing active ingredients to linger in the mouth for an extended period.

26. A use of the dental product according to claim 16 in a treatment of dental caries and improvement of oral health, wherein the dental product is used immediately before using a fluoride containing toothpaste for enhancing the fluoride uptake of fluoride from the toothpaste.

27. A first method of use of the dental product according to claim 16 in the treatment of dental caries comprising the steps of;
consuming a minimum number of dental products in a timeframe of one day (100); and
spreading the consumption over the timeframe (110).

28. A first method of use of the dental product according to claim 27 further comprising the step of:
implementing the spreading periodically over the timeframe (120).

29. A second method of use of the dental product according to claim 16 in the treatment of dental caries comprising the steps of:
consuming the dental product; and
afterwards, brushing teeth with a toothpaste comprising a fluoride compound.
